(19) United States
(12) Patent Application Publication
(10) Pub. No.: US 2010/0329926 A1
(43) Pub. Date: Dec. 30, 2010

(54) EXTRACORPOREAL FLUID CIRCUIT

(75) Inventors: Giuseppe Franzoni, Sassuolo (IT); Luca Caleffi, Carpi (IT); Ranko Sakota, Giugliano in Campania (IT)

Correspondence Address: Pearne & Gordon LLP, 1801 East 9th Street, Suite 1200, Cleveland, OH, 44114-3108, US

(73) Assignee: GAMBRO LUNDIA AB, 22010 Lund (SE)

(21) Appl. No.: 12/918,414

(22) PCT Filed: Feb. 19, 2008
(86) PCT No.: PCT/IB2008/000365

§ 371 Date: Aug. 19, 2010

(51) Int. Cl. A61M 1/36; B01D 63/00

(52) U.S. Cl. 422/44; 210/321.72

(57) ABSTRACT

An extracorporeal blood circuit defines a blood course comprising a first conduit (27) having an upturned U shape forming a curve with a convex external side facing upwards. The circuit has an infusion fluid course with a terminal tract (28) opening into the extracorporeal blood course at said convex external side. The first conduit and the terminal tract are integrated in a single rigid body (20). The circuit enables an optimal mixture between the blood flow and the infusion fluid flow in a relatively-small mixing space.

### BACKGROUND OF THE INVENTION

The invention relates to an extracorporeal fluid circuit and to an extracorporeal blood treatment apparatus using the extracorporeal fluid circuit.

Specifically, though not exclusively, the invention can be usefully applied for infusion of a fluid into an extracorporeal blood flow, such as for example in infusion of a replacement fluid during the course of a hem(dia)filtration treatment, or infusion of a medication during an extracorporeal blood treatment, or infusion of a buffer solution in an AFB (Acetate Free Biofiltration) treatment, and so on.

The prior art comprises WO 02/061318, which describes an apparatus for fluid transport used in the medical field, in which a support element has a U-shaped channel having a semicircular transversal section for housing a tract of flexible transport pipe. WO 02/061318 illustrates another apparatus for fluid transport, in which a support element has a first and a second portions, connected by means of a hinge; both the first and the second portions have a curved channel with a semicircular transversal section; when the support element is closed by rotating the two portions about the hinge, the two portions are symmetrically facing, one on the other, and the two channels meet one another so as to form a single curved closed channel having a circular transversal section, which stably houses a flexible fluid transport pipe.

WO 2007/050211 describes an integrated extracorporeal circuit having a rigid body with a flat surface provided with one or more recesses, covered by a flexible wall. One of the recesses defines, in collaboration with the flexible wall, a gas-liquid separation chamber provided with a microporous hydrophobic membrane vent.

WO 95/17218 describes a medical fluid circuit in which a circuit tray receives and holds in ordered and compact positions all the various parts of the circuit, such as the processor chamber, the fluid containers, the cassettes, fluid transport pipes and so on.

U.S. Pat. No. 5,311,908 illustrates an integrated apparatus for fluid transport. The apparatus comprises a plurality of flexible tubes intercommunicating with one another, all being incorporated in a rigid cassette frame.

EP 568275 describes various embodiments of an extracorporeal blood circuit having an expansion chamber for the separation of air bubbles from the blood flow and an infusion fluid conduit opening onto a blood conduit which carries the blood to the expansion chamber. In one of these examples the blood conduit forms a U-shaped curve and the infusion fluid conduit opens on the external side of the curve.

### SUMMARY OF THE INVENTION

An aim of the present invention is to provide an extracorporeal fluid circuit which can ensure a regular and controlled flow both of the extracorporeal blood and of an infusion fluid which is introduced into the blood itself.

A further aim of the invention is to provide an extracorporeal fluid circuit in which the risk of formation of kinking and other occlusions in the circuit is considerably reduced.

An advantage of the invention is to provide a constructively simple and economical circuit.

A further advantage is to make available a circuit in which the formation of kinking and other occlusions and irregularities are prevented, in particular in a zone of the circuit in which the extracorporeal blood flow receives an infusion fluid flow.

A still further advantage is that it gives rise to a small-size circuit which is easy and immediate to use.

These aims and advantages and more besides are all attained by the invention as it is characterised in one or more of the accompanying claims.

In a specific embodiment of the invention, the fluid circuit comprises an expansion chamber and a tract of tube which are both rigid and which are also rigidly connected to one another. The expansion chamber is configured for gas-liquid separation in a flow of infusion liquid, while the tract of tube is configured for the transport of blood. The fluid circuit comprises a fluid connection which is configured for sending the infusion fluid from the expansion chamber to the tract of tube.

In a specific embodiment, the tract of rigid tube forms a bend and receives the infusion fluid on an external side of the bend. In the contact zone between the infusion fluid and the blood, the direction of the infusion fluid flow can be tangential and equal to the blood flow direction.

In a specific embodiment, the fluid circuit comprises a pump tube configured for coupling with a fluid transport pump. The above-cited pump tube is configured for transporting the infusion fluid from the expansion chamber to the tube tract.

In a specific embodiment, the pump tube is fluidly arranged between the expansion chamber and the tube tract. The pump tube may be supported by the expansion chamber.

In a specific embodiment, the fluid circuit comprises a transport conduit for the infusion fluid which is fluidly arranged between the expansion chamber and the tube tract. The above-cited transport conduit for the infusion fluid is rigid and is further rigidly connected both to the above-mentioned expansion chamber and to the tube tract.

Further characteristics and advantages of the present invention will better emerge from the detailed description that follows, of at least an embodiment of the invention, illustrated by way of non-limiting example in the accompanying figures of the drawings.

### BRIEF DESCRIPTION OF THE DRAWINGS

The description will be made herein below with reference to the accompanying figures of the drawings, which are provided by way of non-limiting example.

FIG. 1 is an extracorporeal blood apparatus comprising a fluid circuit of the invention.

FIG. 2 shows a detail of the fluid circuit of FIG. 1 in greater detail.

FIG. 3 shows a second embodiment of the fluid circuit.

FIG. 4 is a lateral view from the right of FIG. 3.

FIG. 5 is a lateral view from the right of FIG. 4.

FIG. 6 is a view from the bottom of FIG. 5.

FIG. 7 is a view from the top of FIG. 5.

FIG. 8 is a section made according to the plane VIII-VIII of FIG. 3.

FIG. 9 is a section made according to the plane IX-IX of FIG. 5.

FIG. 10 is a section made according to the plane X-X of FIG. 5.

### DETAILED DESCRIPTION

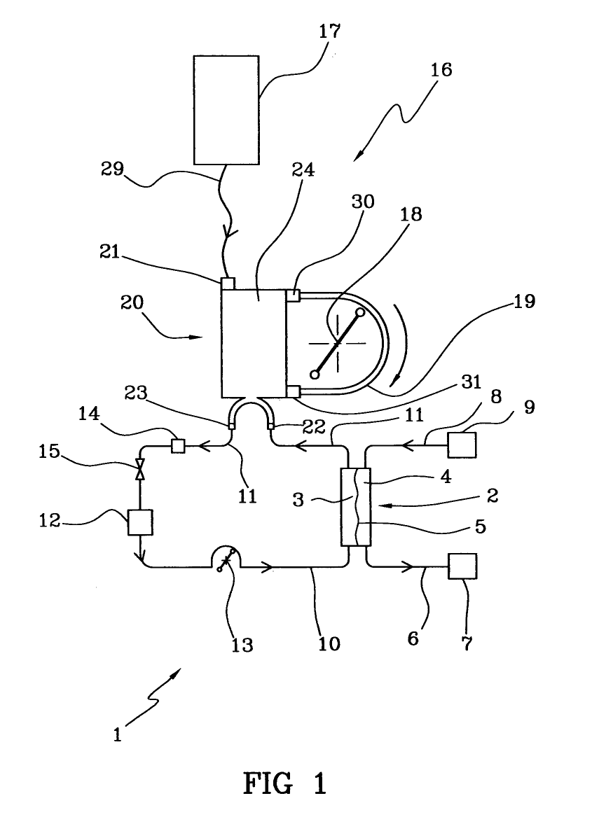

With reference to FIGS. 1, 1 denotes in its entirety an extracorporeal blood treatment apparatus. The extracorporeal blood treatment apparatus 1 comprises a membrane exchanger 2 for an extracorporeal blood treatment. The membrane exchanger 2 can be, for example, a dialyser, a hemofilter, a hemodiafilter, a plasma exchanger, an ultrafilter for congestive hear failure, or another membrane device of known type for performing an extracorporeal treatment. The membrane exchanger 2 has a blood chamber 3 and a fluid chamber 4 which are separated from one another by a semipermeable membrane 5. The extracorporeal blood treatment apparatus 1 comprises a fluid circuit having a discharge line 6 which connects the fluid chamber 4 with a drainage 7. The fluid circuit can optionally have a supply line 8 which connects a treatment fluid source 9 to the fluid chamber 4. The fluid circuit is provided with various elements, not illustrated, such as for example the actuators and sensors with which the fluid circuit of any of the prior-art hemodialysis or hemo(dia)filtration apparatus is provided.

The extracorporeal blood treatment apparatus 1 comprises an extracorporeal fluid circuit connected to the blood chamber 4. The extracorporeal fluid circuit includes a blood path which can have, as in the specific case described herein, an arterial line 10 and a venous line 11. The arterial line 10 is configured for removing the blood from a patient 12 and for sending it on to the blood chamber 3. The venous line 11 is configured for returning the blood treated in the blood chamber 3 to the patient 12. The connection between the patient 12 and the extracorporeal blood path can be realised by any of the vascular access devices of known type.

The extracorporeal blood treatment apparatus 1 optionally comprises a blood pump 13 for moving the blood along the extracorporeal circuit. The blood pump 13 can be operatively associated to the arterial line 10, as in the illustrated example. The extracorporeal circuit can comprise one or more of the various elements (such as for example one or more access sites for sample removal or injection of substances, one or more expansion chambers, one or more air bubble separators, one or more devices for connection to pressure sensors, one or more service lines for performing various services such as supply of priming fluid, introduction of a medical fluid, level regulation in an expansion chamber, etc., portions configured for coupling with hematocrit sensors and/or air bubble sensors and/or blood presence/absence sensors, manual and/or automatic clamps, etc.) with which blood circuits of known type are equipped, such as for example extracorporeal blood circuits used in dialysis apparatus.

In the specific embodiment, and purely by way of example, the following have been indicated: the presence of an expansion chamber 14 arranged on the venous line 11 for air-blood separation, as well as the presence of a block valve 15 (automatically controlled by the control unit of the apparatus 1) arranged on the venous line 11 between the expansion chamber 14 and the patient 12. However, the blood circuit can be provided with other elements (of known type and not illustrated for the sake of simplicity) apart from those indicated.

The extracorporeal fluid circuit is further provided with an infusion system denoted in its entirety by 16. The infusion system 16 is configured for introducing an infusion fluid into the extracorporeal blood path. The infusion fluid can comprise any one of the fluids which, according to the prior art, can be introduced into the extracorporeal blood during an extracorporeal blood treatment. Optionally the infusion fluid can comprise a substitution fluid in a hemo(dia)filtration treatment, or a buffer solution (for example bicarbonate) in an extracorporeal blood treatment (for example a kidney failure treatment).

In the specific embodiment the infusion system 16 is configured for introducing the infusion fluid into the venous line 11. It is however possible for the infusion system 16 to be predisposed for introducing the infusion fluid into the arterial line 10 as well, in addition (simultaneously or alternatedly) or alternatively of the introduction thereof into the venous line 11.

The infusion system 16 optionally comprises an infusion fluid source which, in the example, comprises a batch container 17 of an infusion fluid. The infusion fluid source can be any known-type infusion fluid source, such as for example a source comprising an on-line preparation device of a medical fluid, for instance starting from water and concentrates (such as for example any on-line preparator of substitution fluid used in a hemo(dia)filtration apparatus).

The infusion system 16 optionally comprises an infusion fluid path which connects the infusion fluid source 17 to an infusion zone arranged on a blood path defined by the extracorporeal circuit. The infusion system 16 optionally comprises an infusion fluid pump 18 for moving the infusion fluid from the source 17 to the extracorporeal blood path. The infusion fluid pump 18 can, for example, comprise a tube-deforming pump (peristaltic pump). In the specific case the infusion fluid pump 18 comprises a tube deformation pump of a rotary type. The infusion fluid pump 18 is optionally configured for coupling with a pump tube 19. The pump tube 19 comprises, in the specific case, a tract of curved tube, for example a U-shaped tube, of known type. The pump tube 19 defines a tract of infusion fluid circuit.

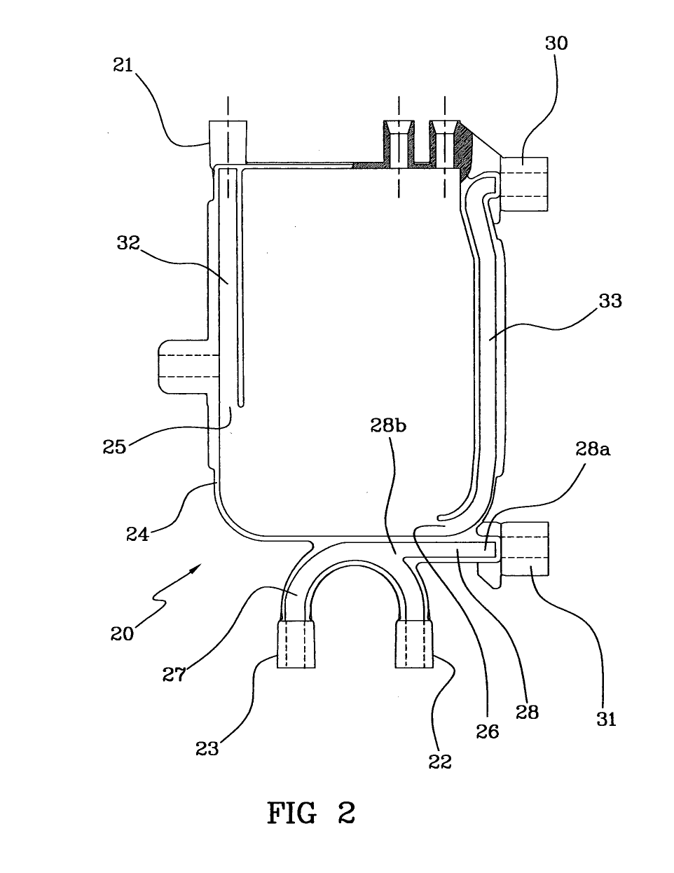

The infusion system 16 comprises a rigid body 20 which is illustrated in greater detail in FIG. 2. The rigid body 20 defines a first fluid passage port 21, a second fluid passage port 22, and a third fluid passage port 23. The first fluid passage port 21 optionally comprises a tubular connector for tubes. This tubular connector for tubes may comprise one of the known tubular connectors configured for removable connections of medical tubes, for example luer coupling, hansen coupling, quick-connective coupling, etc., or for permanent connections of medical tubes, for example by welding or gluing. The second fluid passage port 22 optionally comprises a tubular connector for tubes. This tubular connector for tubes may comprise one of the known tubular connectors configured for removable connections of medical tubes, for example luer coupling, hansen coupling, quick-connective coupling, etc., or for permanent connections of medical tubes, for example by welding or gluing. The third fluid passage port 23 optionally comprises a tubular connector for tubes. This tubular connector for tubes may comprise one of the known tubular connectors configured for removable connections of medical tubes, for example luer coupling, hansen coupling, quick-connective coupling, etc., or for permanent connections of medical tubes, for example by welding or gluing.

The rigid body 20 further defines an expansion chamber 24 having an inlet 25 and an outlet 26. The inlet 25 is optionally arranged above the outlet 26, with reference to a use configuration of the chamber 24. The expansion chamber 24 is provided with a bottom and a top, with reference to the use configuration of the expansion chamber 24. The outlet 26 is optionally arranged on the bottom of the expansion chamber 24. The inlet 25 is optionally arranged at an intermediate level comprises between the bottom and the top. The inlet 25 communicates with the first fluid passage port 21. The first fluid passage port 21 is optionally arranged on a top of the rigid body 20. The first fluid passage port 21 has a longitudinal axis which is optionally directed vertically. The first fluid passage port 21 is optionally upwards-facing.

The expansion chamber 24 is configured for degassing a liquid which flows from the inlet 25 to the outlet 26. In the specific case the liquid to be degassed is the infusion fluid coming from the infusion fluid source 17, as will be more fully described herein below. The expansion chamber 24 is optionally configured for operating in a way such as to define, during a the flow of the liquid to be degassed from the inlet 25 to the outlet 26, a gas-liquid separation level. The upper portion of the expansion chamber, i.e. the portion situated above the liquid level, functions as a gas-accumulation zone, while the lower portion, situated below the liquid level, is full of liquid. The liquid level is predetermined, in a known way, such that the inlet 25 and the outlet 26 are arranged below the level.

It is however possible to have the expansion chamber realised as any of the expansion chambers used in known-type extracorporeal blood circuits or infusion circuits, such as for example an expansion chamber for gas-liquid separation of the complete-filling type with a gas vent provided with a hydrophobic membrane, or an expansion chamber for gas-liquid separation of the hydrophilic type for non-dissolved gas filtration and with a gas vent provided with a hydrophobic membrane, etc.

The rigid body 20 further defines a first fluid passage conduit 27 which extends between the second and the third fluid passage port 22 and 23. The rigid body 20 further defines a second fluid passage conduit 28 having a first end 28a and a second end 28b. The first end 28a is connected, either directly or (as in the illustrated example) indirectly, to the outlet 26 of the expansion chamber 24. The second end 28b may be attached to the first fluid passage conduit 27. The second fluid passage conduit 28 may open into the first fluid passage conduit 27 at an end zone. The end zone gives rise to a mixing zone between the infusion fluid, borne by the second conduit 28, and the extracorporeal blood, borne by the first conduit 27. Optionally, both the first conduit 27 and the second conduit 28 are rigid, as in the illustrated example. It is possible to have one only of the two above-mentioned conduits 27 and 28 rigid, the conduit 27 or the conduit 28.

The blood path comprises a first flexible tube and a second flexible tube, each of which is configured for blood transport. The first flexible tube has an end connected (removably or permanently) to the second fluid passage port 22. The second flexible tube has an end connected (removably or permanently) to the third fluid passage port 23. In the specific case the first and the second flexible tubes are two tracts of the above-cited extracorporeal blood path. Optionally, as in the specific case, the first flexible tube is comprised in an initial tract of the venous line 11 which goes from the blood chamber 3 to the second port 22, while the second flexible tube is comprised in a final tract of the venous line 11 which goes from third port 23 to the patient 12. In this specific case the first conduit 27 substantially forms an intermediate tract of the venous line 11 comprised between the above-mentioned initial and final tracts.

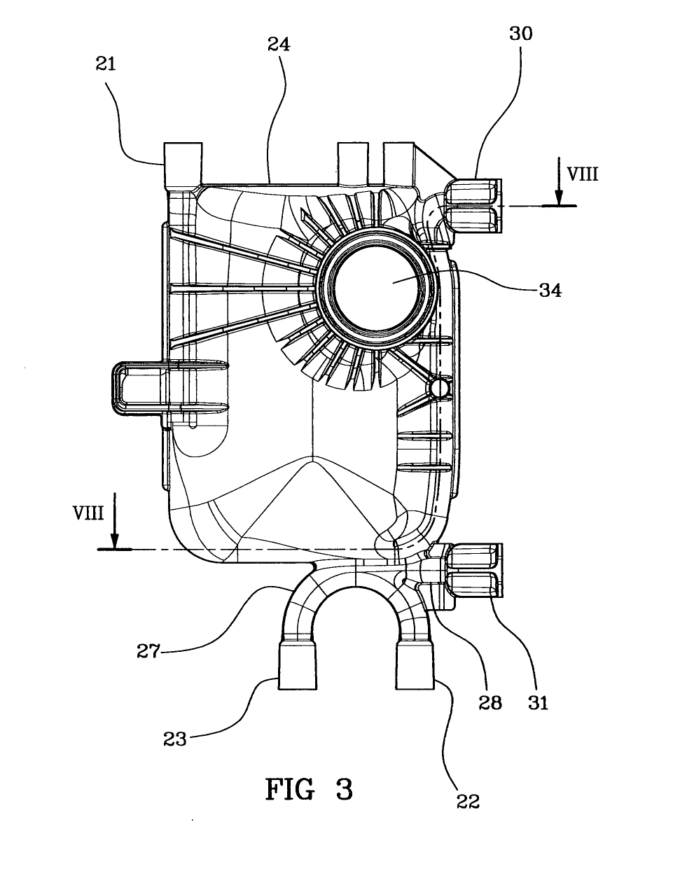

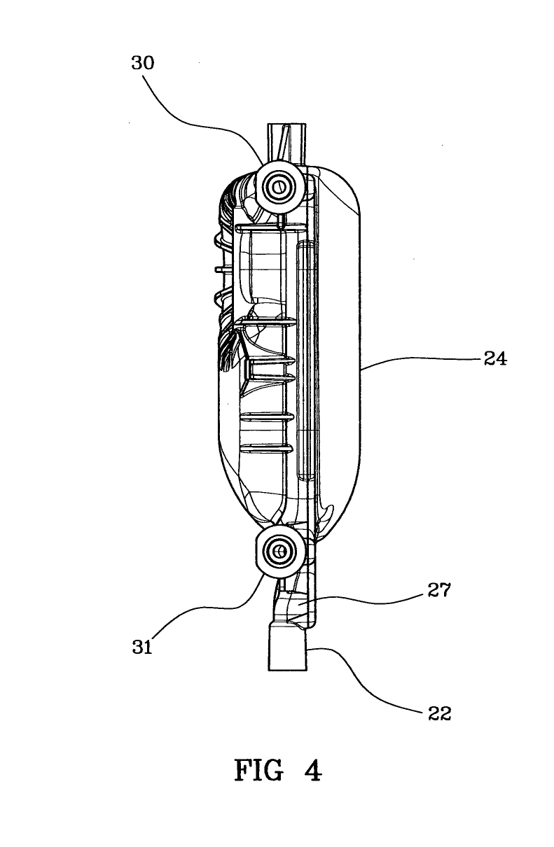

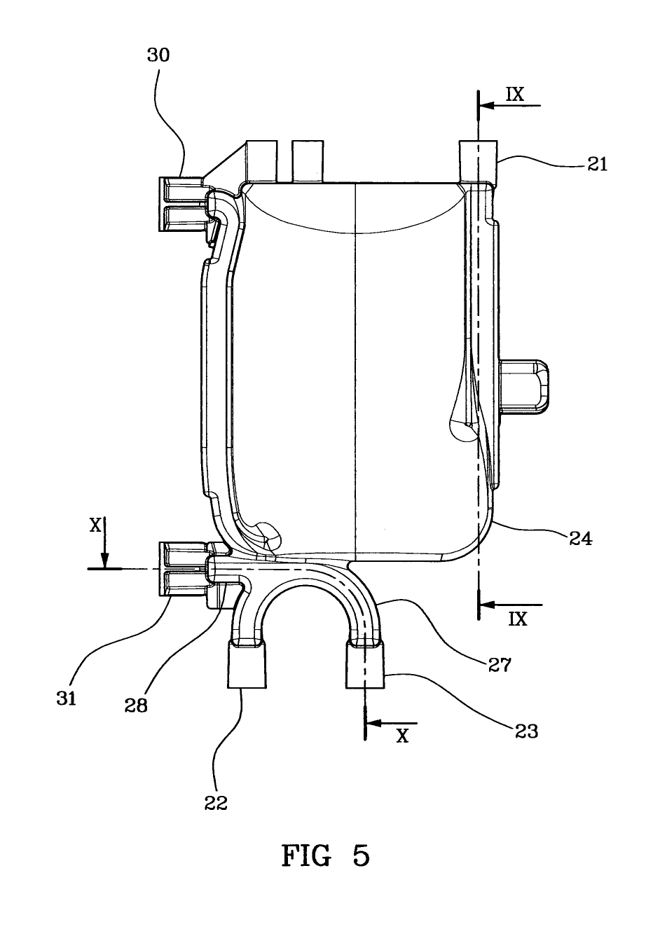

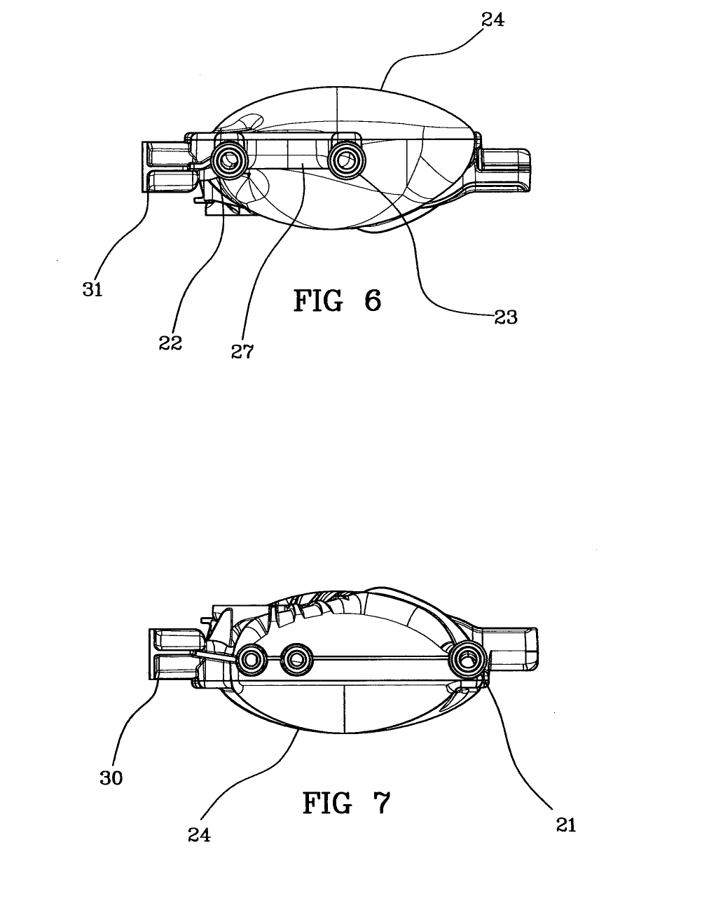

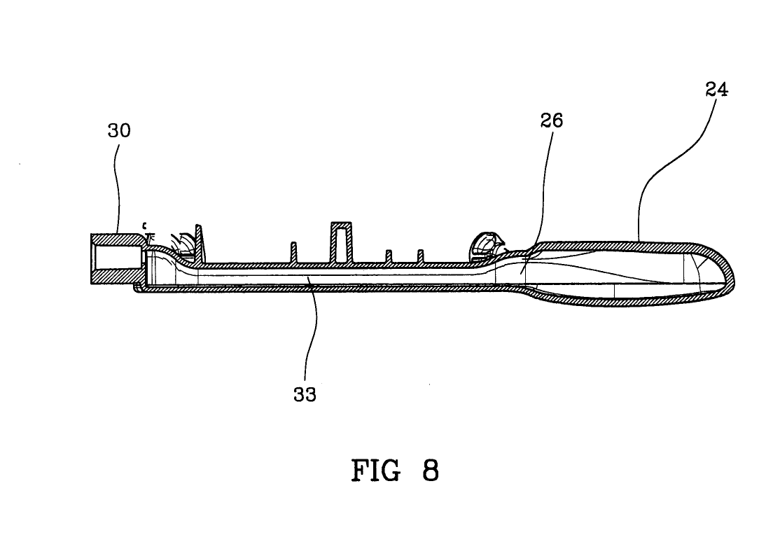

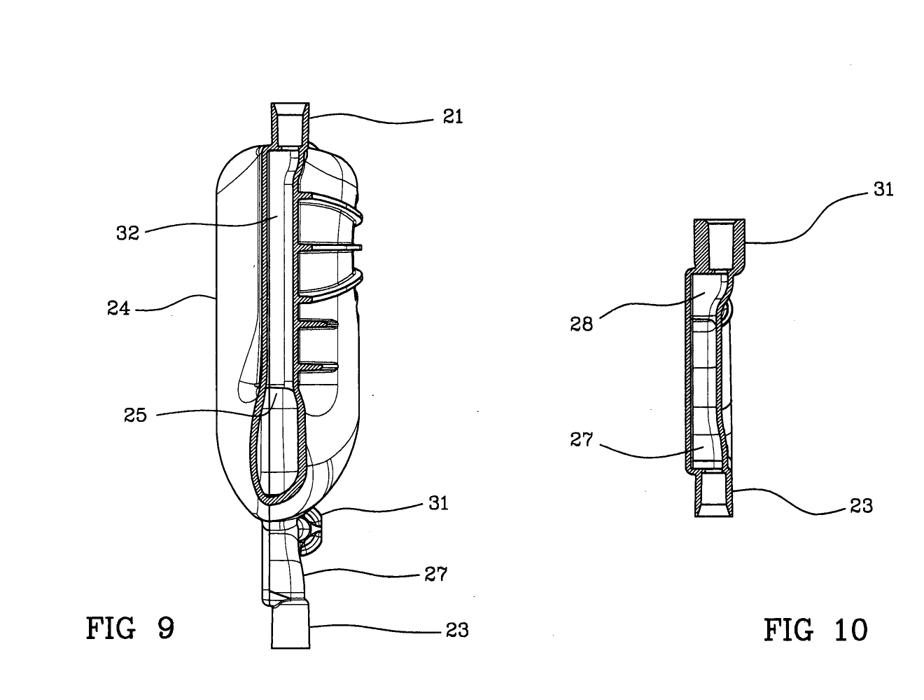

In a further embodiment (see FIGS. 2 to 10, in which the same elements as those in FIGS. 1 and 2 have been denoted using the same numbers) the extracorporeal fluid circuit (which can be associated to the apparatus 1 of FIG. 1) further comprises a pressure sensor 34 for emitting a signal indicating the pressure in the expansion chamber 24. The pressure sensor 34 can comprise any known pressure sensor used in a fluid circuit for medical use. In particular the pressure sensor 34 may be an elastically-deformable membrane sensor having an internal side which faces towards the inside of the expansion chamber 24 and an external side which communicates with a pressure transducer (of known type and not illustrated) connected to the control unit of the treatment apparatus 1. The pressure transducer may be predisposed such as to be able to measure the pressure in the inside of the expansion chamber 24.

The elastically-deformable membrane sensor 34 may be solidly associated to the body of the expansion chamber 24. In particular, the membrane can have the edge thereof tightly engaged between two half-shells which are part of the body of the expansion chamber 24. It is possible to use other types of known pressure sensors, such as for example the pressure sensors already used in the medical field for measuring the pressure in extracorporeal blood circuits. In particular it is possible to use a pressure sensor comprising a service line connecting the expansion chamber 24 with a pressure transducer—in turn connected to the control unit of the treatment apparatus—via the interpositioning of a transducer-protector device having a hydrophobic membrane (also known as a blood catcher).

The extracorporeal fluid circuit comprises a blood course tract with is realised by a flexible conduit to which a rigid conduit follows, which in turn is followed by a flexible conduit. The inlet zone of the infusion fluid into the blood course is situated at a blood course tract defined by at least a rigid wall. In substance the blood course has a tract which is defined by at least a rigid wall and which is preceded and followed, respectively, by two tracts defined by at least a flexible wall.

The infusion fluid course terminates (optionally with a terminal tract which is at least partly rigid) at a tract of blood course which is at least partly rigid. The at least partly-rigid tract of blood course can, in the specific case, form a curve. The at least partly-rigid tract of blood course can, as in the specific case, be integrated with an expansion chamber configured for gas-liquid separation in the flow of infusion fluid before the flow itself opens into the blood course at the above-mentioned at least partly-rigid tract of blood circuit; the at least partly-rigid tract of blood course and the expansion chamber for the infusion fluid are integrated in a rigid body; the rigid body can optionally support a pump tube 19 for moving the infusion fluid.

The infusion system 16 comprises a third flexible tube 29, configured for the infusion fluid transport. The third flexible tube 29 has a first end connected to the first fluid passage port 21 and a second end connected to the infusion fluid source 17. The third flexible tube 29 is configured to fluidly connect the infusion fluid source with the expansion chamber 24 defined by the rigid body 20. The third flexible tube 29 and the batch container 27 of the infusion fluid are disposable elements, optionally made of plastic.

The connection between the infusion fluid source and the rigid body 20 (in particular with the expansion chamber 24) can be made in another way, for example by means of a non-disposable conduit, or by means of a rigid conduit (disposable or not), or by means of the direct connection of the first port 21 with an on-line supply system of infusion fluid, etc.

The pump tube 19 can define, as in the specific embodiment described herein, a fluid circuit comprised between the outlet 26 of the expansion chamber 24 and the first end 28a of the second conduit 28. The pump tube 19 optionally has two opposite ends supported by the rigid body 20. In the specific embodiment the rigid body 20 defines a fourth fluid passage port 30 and a fifth fluid passage port 31. The fourth and fifth fluid passage ports 30 and 31 each comprise a tubular connector for coupling (both mechanical, for example by welding or gluing or a fluid connection) with an end of a tube. In the specific case the two opposite ends of the pump tube 19 are supported by the fourth and fifth fluid passage port 30 and 31. In particular the two opposite ends of the pump tube 19 are in fluid connection with the fourth and fifth fluid passage port 30 and 31. The fourth fluid passage port 30 is, in the specific embodiment, in fluid connection (direct or, as in the described embodiment, indirect), with the outlet 26 of the expansion chamber 24. The fifth fluid passage port 31 is, in the present embodiment, in fluid connection (indirect or, as in the present embodiment, direct) with the first end 28a of the second fluid passage 28.

The rigid body 20 defines a third fluid passage conduit 32 extended between the first fluid passage port 21 and the inlet 25 of the expansion chamber 24. The third fluid passage conduit 32 extends on a first side of the expansion chamber 24 in a prevalently vertical direction, with reference to a use configuration of the expansion chamber (the configuration in which the plane of FIG. 2 is a vertical elevation plane). The first side of the expansion chamber 24 extends between the bottom and the top of the expansion chamber 24 itself. In the use configuration of the expansion chamber 24 the rotation axis of the infusion fluid pump 18 is arranged horizontally, and the pump tube 19 extends on a vertical lie plane.

The rigid body 20 defines a fourth fluid passage conduit 33 which defines a fluid circuit (for the infusion fluid in this case) comprised between the outlet 26 and the expansion chamber 24 and the first end 28a of the second fluid passage conduit 28. The pump tube 19 is fluidically interposed between the fourth fluid passage conduit 33 and the second fluid passage conduit 28. In the specific case the fourth fluid passage conduit 33 defines a fluid course comprised between the outlet 26 of the expansion chamber 24 and the pump tube 19.

The fourth fluid passage conduit 33 extends on a second side of the expansion chamber 24 in a prevalently vertical direction, with reference to the use configuration of the expansion chamber. The second side of the expansion chamber 24 extends between the bottom and the top of the chamber. The second side of the expansion chamber 24 is opposite the above-mentioned first side of the expansion chamber 24.

The third and the fourth fluid passage conduit, respectively 32 and 33, are arranged on two opposite sides of the expansion chamber 24; optionally these opposite sides each extend between the bottom and the top of the expansion chamber 24. The third and fourth fluid passages, respectively 32 and 33, are both extended in a prevalently vertical direction, with reference to the use configuration of the expansion chamber. In the specific case the pump tube 19 defines a fluid course comprised between the fourth fluid passage conduit 33 and the first end 28a of the second fluid passage conduit 28.

The rigid body 20 integrates the expansion chamber 24 (which serves in particular for the gas-liquid separation of the infusion fluid), the first conduit 27 (which serves in particular for connecting up two portions of the blood course realised at least in part with flexible tubes) and the second conduit 28 (which serves in particular for transporting the infusion fluid from the expansion chamber 24 of the first conduit 27), such that the three elements are solidly connected to one another. In particular the reciprocal connection between these three elements is configured such that the reciprocal positioning thereof is predefined and stable. This can enable, for example, easy manoeuvring of the elements, such as in particular to improve the ease of the mounting operations onto the treatment apparatus of an extracorporeal circuit provided with an infusion system.

The fact that the mixing zone between the infusion fluid and the extracorporeal blood is realised by a rigid structure incorporated in an expansion chamber of the infusion fluid, reduces the risk of irregularity in the flows of the various liquids involved (blood, infusion fluid and mixture thereof) and/or occluding the transport conduits and/or deforming the conduits themselves (deformations such as, for example, kinking of flexible tubes).

The optional fact that the rigid body is the support of the infusion fluid pump tube enables a reduction to be made in the overall size of infusion system and facilitates the mounting thereof in an operative position.

The rigid body 20 can optionally be made of plastic. The rigid body 20 can be realised in various ways, such as for example by assembly (gluing, welding, etc.) of two half-shells; the half-shells can be realised, for example, by moulding of plastic material.

The first fluid passage conduit 27 optionally has a longitudinal axis which forms a curve with a concave internal side facing downwards, with reference to the use configuration of the expansion chamber 24. The second fluid passage conduit 28 optionally has a substantially straight longitudinal axis with a horizontal direction, with reference to the use configuration of the expansion chamber. The first and second fluid passage conduits 27 and 28 optionally have two longitudinal axes which may be substantially tangential to one another. The first and second fluid passage conduits 27 and 28 are optionally arranged below the expansion chamber 24, with reference to the use configuration of the expansion chamber.

The fluid circuit described herein with reference to FIGS. 1 and 2 comprises an extracorporeal blood course and an infusion fluid course. The blood course can comprise, as in the specific case, an extracorporeal blood circuit configured for connecting a patient 12 with a blood chamber 3 of a membrane device 2 for extracorporeal blood treatment. The infusion fluid course opens into the blood course.

The blood course comprises a rigid tube which forms a curve. The above-cited rigid tube can comprise, for example, the above-described first conduit 27. The curve formed by the rigid tube has a convex external side which is at least part upward-facing, with reference to a use configuration of the rigid tube (the configuration in which the plane of FIG. 2 is a vertical elevation plane).

The infusion fluid course can comprise, for example, the above-described second conduit 28. The infusion fluid course can comprise any part or the whole course formed by the above-described infusion fluid system 16. The infusion fluid course opens into the blood course at the above-described external convex side. The curve formed by the rigid tube has an upturned-U shape, with reference to the use configuration of the rigid tube.

The infusion fluid course ends in the blood course in an end zone. The infusion fluid course and the blood course are conformed and reciprocally arranged such that in the end zone the movement direction of the infusion fluid includes at least a component going in the same direction as the blood movement. In particular, in the end zone the movement direction of the infusion fluid is tangential to the movement direction of the blood.

The infusion fluid course has a terminal tract that ends in the blood course with a substantially horizontal direction, with reference to the use configuration of the rigid tube. The terminal tract of the infusion fluid course can be realised, for example, by the second conduit 28. The terminal tract of the infusion fluid course, i.e. the tract which ends in the blood course, can be rigid.

The terminal tract of the infusion fluid course and the curved rigid tube of the blood course are integrated in a single rigid body, which in the example comprises the rigid body 20 described above.

In the specific case the curved rigid tube (i.e. the first conduit 27) has a straight transversal section with an average diameter comprised between 3 and 15 millimetres. In particular the curved rigid tube can have a straight transversal section having an average diameter comprised between 3 and 8 millimetres, for example about 5 millimetres (as in the specific embodiment of FIG. 2). In the end zone, the curved rigid tube optionally has a radius of curvature comprised between 10 and 40 millimetres. In particular the rigid curved tube can have, in the end zone, a radius of curvature comprised between 10 and 25 millimetres, for example about 15 millimetres (as in the specific example of FIG. 2).

The rigid curved tube has a straight transversal section having a determined average diameter D, and further has, in the end zone, a determined radius of curvature R. The R/D ratio between the radius of curvature R and the average diameter D can be comprised between 1.5 and 7.5. In particular the R/D ration between the radius of curvature R and the average diameter D can be comprised between 2 and 5, for example about 3 (as in the specific example of FIG. 2).

The curved rigid tube can have a straight transversal section having a substantially constant diameter. The curved rigid tube can be extended along an arc, for example an arc of circumference, which subtends an angle greater than 60 degrees. In the specific case of FIG. 2, the curved rigid tube extends along an arc of circumference which has an angle of about 180 degrees. The curved rigid tube may extend along an arc (of circumference) which subtends an angle greater than 90 degrees. The curved rigid tube may extend along an arc (of circumference) which subtends an angle comprised between 90 and 270 degrees. The curved rigid tube may extend along an arc (of circumference) which, downstream of the end zone of the infusion fluid course, subtends an angle greater than 30 or 45 or 60 degrees. In the specific case of FIG. 2, the curved rigid tube extends along an arc of circumference which, downstream of the end zone of the infusion fluid course, subtends an angle of about 90 degrees.

The curve rigid tube and the expansion chamber are separated from one another by an impermeable and rigid wall which defines the bottom of the expansion chamber.

The particular structure and configuration of the curved rigid tube included in the blood course (which in the special embodiment described herein comprises the first conduit 27) and the terminal tract of the infusion fluid course (which in the specific case comprises the second conduit 28) enables an optimal mixture between the two flows in a relatively-small mixing space.

It is also reduced the risk of forming turbulence, or bubbles, or foam, or whirls, or other irregularities in the flow, especially after the mixing zone. A further advantage is a low risk of damages to the blood.

1-60. (canceled)

61. A fluid circuit comprising an extracorporeal blood course and an infusion fluid course, wherein said infusion fluid course ends in said extracorporeal blood course in an end zone; said extracorporeal blood course comprising at least a rigid tube having a straight transversal section with a substantially constant diameter, said rigid tube forming a curve having a convex external side; said infusion fluid course ending in said extracorporeal blood course at said convex external side; said convex external side at least partly facing upwards, with reference to a use configuration of said rigid tube, said infusion fluid course comprises an expansion chamber, said rigid tube being rigidly connected below said expansion chamber with reference to a use configuration of said expansion chamber.

62. The circuit of claim 61, wherein said infusion fluid course and said extracorporeal blood course are configured such that in said end zone the movement direction of the infusion fluid has at least a movement component going in the movement direction of the blood.

63. The circuit of claim 61, wherein said infusion fluid course has a terminal tract which ends in said extracorporeal blood course, said terminal tract being rigidly connected to said expansion chamber.

64. The circuit of claim 61, wherein said infusion fluid course comprises a pump tube configured for coupling with a tube-deforming pump, said pump tube defining a fluid course comprised between said outlet of said expansion chamber and said extracorporeal blood course.

65. The circuit of claim 61, wherein said infusion fluid course comprises a pump tube configured for coupling with a tube-deforming pump, said pump tube having two ends rigidly connected to said rigid tube.

66. The circuit of claim 61, wherein said rigid tube has a first end arranged downstream of said end zone; said first end comprising a first fluid port which at least partly faces downwards, with reference to a use configuration of said rigid tube.

67. The circuit of claim 66, comprising a first flexible tube connected to said first fluid port.

68. The circuit of claim 61, wherein said rigid tube has a second end arranged upstream of said end zone; said second end having a second fluid port which at least partly faces downwards, with reference to a use configuration of said rigid tube.

69. The circuit of claim 68, comprising a second flexible tube connected to said second fluid port.

70. The circuit of claim 61, wherein said extracorporeal blood course comprises a blood pump tube configured for coupling with a tube-deforming pump, said blood pump tube being arranged upstream of said end zone.

71. The circuit of claim 61, wherein said extracorporeal blood course comprises a blood chamber of a membrane exchanger, said blood chamber being separated from a fluid chamber of said membrane exchanger by a semipermeable membrane, said end zone being arranged downstream of said blood chamber.

72. The circuit of claim 61, wherein said curve formed by said rigid tube has an upturned U-shape.

73. The circuit of claim 61, wherein said infusion fluid course and said extracorporeal blood course are configured such that in said end zone the movement direction of the infusion fluid is substantially tangential to the movement direction of the blood.

74. The circuit of claim 61, wherein said infusion fluid course has a terminal tract which ends in said extracorporeal blood course and which has a substantially horizontal direction, with reference to a use configuration of said rigid tube.

75. The circuit of claim 61, wherein said infusion fluid course has a terminal tract which ends in said extracorporeal blood course, said terminal tract being rigid.

76. The circuit of claim 75, wherein said rigid terminal tract of said infusion fluid course and said curved rigid tube of said extracorporeal blood course are integrated in a single rigid body.

77. The circuit of claim 61, wherein said curved rigid tube has a straight transversal section having an average diameter comprised between 3 and 15 millimetres.

78. The circuit of claim 61, wherein said curved rigid tube has, in said end zone, a radius of curvature comprised between 10 and 40 millimetres.

79. The circuit of claim 61, wherein said curved rigid tube has a straight transversal section with a determined average diameter; said curved rigid tube further having, in said end, a determined radius of curvature; the ratio between said determined radius of curvature and said determined average diameter being comprised between 1.5 and 7.5.

80. The circuit of claim 61, wherein said curved rigid tube extends along an arc which subtends an angle greater than 60 degrees.

81. The circuit of claim 61, wherein said curved rigid tube extends, downstream of said end zone, along an arc which subtends an angle greater than 30 degrees.

82. The circuit of claim 61, wherein said extracorporeal blood course comprises a first flexible tube and a second flexible tube; said first flexible tube having an end which is connected to said curved rigid tube; said second flexible tube having an end which is connected to said curved rigid tube.

83. The circuit of claim 61, wherein said infusion fluid course comprises a source of the infusion fluid, said source comprising a batch container of the infusion fluid.

84. The circuit of claim 61, wherein said curved rigid tube has a longitudinal axis which forms a curve with a concave internal side facing downwards with reference to a use configuration of said curved rigid tube.

85. The circuit of claim 61, wherein said infusion fluid course has a terminal tract which ends in said extracorporeal blood course; said terminal tract of said infusion fluid course and said curved rigid tube having two longitudinal axes which are substantially tangential to one another.

86. A fluid circuit comprising an extracorporeal blood course and an infusion fluid course; said extracorporeal blood course comprising at least a rigid tube which forms a curve; said infusion fluid course ending in said extracorporeal blood course in an end zone included in said curve; said infusion fluid course comprising an expansion chamber arranged upstream of said end zone; said curve and said expansion chamber being separated from one another by an impermeable and rigid wall; said expansion chamber communicating with said end zone through a fluid passage conduit, wherein said infusion fluid course comprises a pump tube configured for coupling with a tube-deforming pump, said pump tube defining a fluid course comprised between said outlet of said expansion chamber and said extracorporeal blood course.

87. The circuit of claim 86, wherein said curve has a convex external side; said infusion fluid course ending in said extracorporeal blood course at said convex external side.

88. The circuit of claim 87, wherein said convex external side at least partly faces upwards, with reference to a use configuration of said rigid tube and said expansion chamber.

89. The circuit of claim 86, wherein said infusion fluid course and said extracorporeal blood course are configured such that in said end zone the movement direction of the infusion fluid has at least a movement component going in the movement direction of the blood.

90. The circuit of claim 86, wherein said rigid tube is rigidly connected to said expansion chamber.

91. The circuit of claim 86, wherein said infusion fluid course has a terminal tract which ends in said extracorporeal blood course, said terminal tract being rigidly connected to said expansion chamber.

92. The circuit of claim 86, wherein said curved rigid tube is arranged below said expansion chamber, with reference to a use configuration of said expansion chamber.

93. The circuit of claim 86, wherein said infusion fluid course comprises a pump tube configured for coupling with a tube-deforming pump, said pump tube having two ends rigidly connected to said rigid tube.

94. The circuit of claim 86, wherein said rigid tube has a first end arranged downstream of said end zone; said first end comprising a first fluid port which at least partly faces downwards, with reference to a use configuration of said rigid tube.

95. The circuit of claim 94, comprising a first flexible tube connected to said first fluid port.

96. The circuit of claim 86, wherein said rigid tube has a second end arranged upstream of said end zone; said second end having a second fluid port which at least partly faces downwards, with reference to a use configuration of said rigid tube.

97. The circuit of claim 96, comprising a second flexible tube connected to said second fluid port.

98. The circuit of claim 86, wherein said extracorporeal blood course comprises a blood pump tube configured for coupling with a tube-deforming pump, said blood pump tube being arranged upstream of said end zone.

99. The circuit of claim 86, wherein said extracorporeal blood course comprises a blood chamber of a membrane exchanger, said blood chamber being separated from a fluid chamber of said membrane exchanger by a semipermeable membrane, said end zone being arranged downstream of said blood chamber.

100. The circuit of claim 86, wherein said curve formed by said rigid tube has an upturned U-shape.

101. The circuit of claim 86, wherein said infusion fluid course and said extracorporeal blood course are configured such that in said end zone the movement direction of the infusion fluid is substantially tangential to the movement direction of the blood.

102. The circuit of claim 86, wherein said infusion fluid course has a terminal tract which ends in said extracorporeal blood course and which has a substantially horizontal direction, with reference to a use configuration of said rigid tube.

103. The circuit of claim 86, wherein said infusion fluid course has a terminal tract which ends in said extracorporeal blood course, said terminal tract being rigid.

104. The circuit of the claim 103, wherein said rigid terminal tract of said infusion fluid course and said curved rigid tube of said extracorporeal blood course are integrated in a single rigid body.

105. The circuit of claim 86, wherein said curved rigid tube has a straight transversal section having an average diameter comprised between 3 and 15 millimetres.

106. The circuit of claim 86, wherein said curved rigid tube has, in said end zone, a radius of curvature comprised between 10 and 40 millimetres.

107. The circuit of claim 86, wherein said curved rigid tube has a straight transversal section with a determined average diameter; said curved rigid tube further having, in said end, a determined radius of curvature; the ratio between said determined radius of curvature and said determined average diameter being comprised between 1.5 and 7.5.

108. The circuit of claim 86, wherein said curved rigid tube has a straight transversal section with a substantially constant diameter.

109. The circuit of claim 86, wherein said curved rigid tube extends along an arc which subtends an angle greater than 60 degrees.

110. The circuit of claim 86, wherein said curved rigid tube extends, downstream of said end zone, along an arc which subtends an angle greater than 30 degrees.

111. The circuit of claim 86, wherein said extracorporeal blood course comprises a first flexible tube and a second flexible tube; said first flexible tube having an end which is connected to said curved rigid tube; said second flexible tube having an end which is connected to said curved rigid tube.

112. The circuit of claim 86, wherein said infusion fluid course comprises a source of the infusion fluid, said source comprising a batch container of the infusion fluid.

113. The circuit of claim 86, wherein said infusion fluid course comprises a pump tube configured for coupling with a tube-deforming pump, said pump tube having two ends rigidly connected to said curved rigid tube.

114. The circuit of claim 86, wherein said curved rigid tube has a longitudinal axis which forms a curve with a concave internal side facing downwards with reference to a use configuration of said curved rigid tube.

115. The circuit of claim 86, wherein said infusion fluid course has a terminal tract which ends in said extracorporeal blood course; said terminal tract of said infusion fluid course and said curved rigid tube having two longitudinal axes which are substantially tangential to one another.

116. An extracorporeal blood treatment apparatus, comprising:
a membrane exchanger for extracorporeal blood treatment, said membrane exchanger having a blood chamber and a fluid chamber which are separated from one another by a semipermeable membrane;
a fluid circuit made according to claim 86, said extracorporeal blood course being connected to said blood chamber.
